(19) United States
(12) Patent Application Publication
(10) Pub. No.: US 2025/0381309 A1
(43) Pub. Date: Dec. 18, 2025

(54) EDIBLE CONDUCTIVE STRUCTURE

(71) Applicant: NTT. INC, Tokyo (JP)

(72) Inventors: Kenta Fukada, Tokyo (JP); Takuro Tajima, Tokyo (JP); Michiko Seyama, Tokyo (JP)

(21) Appl. No.: 18/865,880

(22) PCT Filed: Jul. 28, 2022
(86) PCT No.: PCT/JP2022/029120

§ 371 Date: Nov. 14, 2024

(51) Int. Cl. A61K 49/00; A61B 5/1473

(52) U.S. Cl. CPC A61K 49/00; A61B 5/1473

(57) ABSTRACT

This edible conductive structure includes a conductive layer including an edible conductive material and a hydrophobic layer including an edible hydrophobic material. The conductive layer and the hydrophobic layer are alternately stacked. The lowermost layer and the uppermost layer (surface layer) can be the hydrophobic layer. The conductive material can be, for example, an edible metal foil such as gold foil or silver foil. The hydrophobic material can be composed of, for example, an organogel obtained by mixing beeswax and olive oil.

### CROSS-REFERENCE TO RELATED APPLICATIONS

This application is a national phase entry of PCT Application No. PCT/JP2022/029120, filed on Jul. 28, 2022, which application is hereby incorporated herein by reference.

### TECHNICAL FIELD

The present invention relates to an edible conductive structure.

### BACKGROUND

Various edible conductive structures have been developed for analysis of a target substance in a living body, expression of a specific function in a living body, and the like. For example, a conductive adhesive material in which activated carbon, beeswax, and vegetable oil, which are edible materials, are mixed has been proposed (Non Patent Literature 1). This material is liquefied by heating (100° C.) and gelatinized by cooling, and achieves conductivity with a resistivity of 100 Ω·cm by setting the mixing amount of activated carbon to about 40%. Since the gel has viscosity, it is an edible conductive adhesive oleogel, and is intended for application to edible electronics.

In addition, a conductive wiring in which gold having a thickness of 100 nm is formed on rice paper has been proposed (Non Patent Literature 2). This technique is intended for application to edible electronics.

### CITATION LIST

### Non Patent Literature

Non Patent Literature 1: P. Cataldi et al., “An Electrically Conductive Oleogel Paste for Edible Electronics”, Advanced Functional Materials, 2113417, 2022.
Non Patent Literature 2: W. Xu et al., “Food-Based Edible and Nutritive Electronics”, Advanced Functional Materials, 1700181, 2017.

### SUMMARY

### Technical Problem

However, in the technique of Non Patent Literature 1, waterproofness can be expected because wax or oil is used, but activated carbon is used as a conductive material, and the conductivity of the activated carbon itself is lower than that of graphite, metal, or the like, so that the conductivity is low.

In addition, in the technique of Non Patent Literature 2, a gold thin film is used as a conductive material, but the thickness is about 100 to 200 nm, and the resistance is high due to the skin effect. In addition, the rice paper serving as a substrate is considered to be water-absorbing, and it is unclear whether the conductivity can be maintained in water.

The edible electronics is required to operate in a body (for example, in gastric fluid or intestinal fluid) or in an environment (in water or soil), and it is important to develop a material having both waterproofness and conductivity by a food material, but the above-described conventional technique alone may be insufficient for such use applications.

Embodiments of the present invention has been made to solve the above problem, and an object is to provide an edible conductive structure having both waterproofness and conductivity, which can be used in a body or in an environment.

### Solution to Problem

The edible conductive structure according to embodiments of the present invention includes a conductive layer including an edible conductive material and a hydrophobic layer including an edible hydrophobic material, and the conductive layer and the hydrophobic layer are alternately stacked.

### Advantageous Effects of Invention

As described above, according to the present invention, since the conductive layer including an edible conductive material and the hydrophobic layer including an edible hydrophobic material are alternately stacked, it is possible to provide an edible conductive structure having both waterproofness and conductivity, which can be used in the body or in the environment.

### BRIEF DESCRIPTION OF THE DRAWINGS

FIG. 1 is a cross-sectional diagram illustrating a configuration of an edible conductive structure according to an embodiment of the present invention.

FIG. 2A is a photograph illustrating a state of an edible conductive structure in an intermediate process for describing a method for manufacturing the edible conductive structure according to the embodiment of the present invention.

FIG. 2B is a photograph illustrating a state of an edible conductive structure in an intermediate process for describing a method for manufacturing the edible conductive structure according to the embodiment of the present invention.

FIG. 2C is a photograph illustrating a state of an edible conductive structure in an intermediate process for describing a method for manufacturing the edible conductive structure according to the embodiment of the present invention.

FIG. 2D is a photograph illustrating a state of an edible conductive structure in an intermediate process for describing a method for manufacturing the edible conductive structure according to the embodiment of the present invention.

FIG. 2E is a photograph illustrating a state of an edible conductive structure in an intermediate process for describing a method for manufacturing the edible conductive structure according to the embodiment of the present invention.

FIG. 2F is a photograph illustrating a state of an edible conductive structure in an intermediate process for describing a method for manufacturing the edible conductive structure according to the embodiment of the present invention.

FIG. 2G is a photograph illustrating a state of an edible conductive structure in an intermediate process for describing a method for manufacturing the edible conductive structure according to the embodiment of the present invention.

FIG. 2H is a photograph illustrating a state of an edible conductive structure in an intermediate process for describing a method for manufacturing the edible conductive structure according to the embodiment of the present invention.

FIG. 3A is a characteristic diagram illustrating a relationship between a resistance value and a thickness of a wiring-shaped edible conductive structure produced using an edible gold foil and the number of stacked layers.

FIG. 3B is a characteristic diagram illustrating a relationship between a resistance value and a thickness of a wiring-shaped edible conductive structure produced using an edible silver foil and the number of stacked layers.

FIG. 4A is an explanatory diagram illustrating a state of a contact angle on a wiring structure by the edible conductive structure according to the embodiment.

FIG. 4B is a characteristic diagram illustrating a result of measuring a change in resistance value when a wiring structure is formed on a sheet substrate made of gelatin and immersed in water.

FIG. 5A is a photograph illustrating a state of an electronic component to which an edible conductive structure in an intermediate process is applied for describing a method for manufacturing an electronic component to which the edible conductive structure according to the embodiment of the present invention is applied.

FIG. 5B is a photograph illustrating a state of an electronic component to which an edible conductive structure in an intermediate process is applied for describing a method for manufacturing an electronic component to which the edible conductive structure according to the embodiment of the present invention is applied.

FIG. 5C is a photograph illustrating a state of an electronic component to which an edible conductive structure in an intermediate process is applied for describing a method for manufacturing an electronic component to which the edible conductive structure according to the embodiment of the present invention is applied.

FIG. 5D is a photograph illustrating a state of an electronic component to which an edible conductive structure in an intermediate process is applied for describing a method for manufacturing an electronic component to which the edible conductive structure according to the embodiment of the present invention is applied.

FIG. 5E is a photograph illustrating a state of an electronic component to which an edible conductive structure in an intermediate process is applied for describing a method for manufacturing an electronic component to which the edible conductive structure according to the embodiment of the present invention is applied.

FIG. 5F is a photograph illustrating a state of an electronic component to which an edible conductive structure in an intermediate process is applied for describing a method for manufacturing an electronic component to which the edible conductive structure according to the embodiment of the present invention is applied.

FIG. 5G is a photograph illustrating a state of an electronic component to which an edible conductive structure in an intermediate process is applied for describing a method for manufacturing an electronic component to which the edible conductive structure according to the embodiment of the present invention is applied.

FIG. 6A is a photograph illustrating a process of decomposition of the edible conductive structure according to the embodiment of the present invention.

FIG. 6B is a photograph illustrating a process of decomposition of the edible conductive structure according to the embodiment of the present invention.

FIG. 6C is a photograph illustrating a process of decomposition of the edible conductive structure according to the embodiment of the present invention.

FIG. 6D is a photograph illustrating a method of manufacturing the edible conductive structure according to the embodiment of the present invention.

### DETAILED DESCRIPTION OF ILLUSTRATIVE EMBODIMENTS

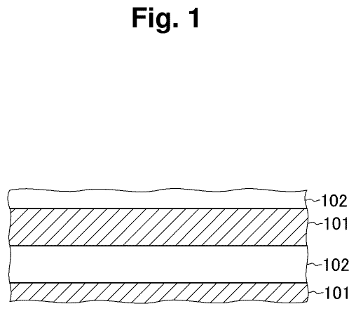

Hereinafter, an edible conductive structure according to an embodiment of the present invention will be described with reference to FIG. 1. The edible conductive structure includes a conductive layer 101 including an edible conductive material and a hydrophobic layer 102 including an edible hydrophobic material. The conductive layer 101 and the hydrophobic layer 102 are alternately stacked. The lowermost layer and the uppermost layer (surface layer) can be the hydrophobic layer 102.

The conductive material can be, for example, an edible metal foil such as gold foil or silver foil. The hydrophobic material can be composed of, for example, an organogel obtained by mixing beeswax and olive oil. In addition, the hydrophobic material can be fat and oil such as fat of meat, lard, corn oil, soybean oil, rapeseed oil, chocolate, butter, and margarine. In addition, the hydrophobic layer 102 is composed of a hydrophobic material to which a conductive material such as a powder of activated carbon or a metal such as zinc, iron, copper, silver, or gold is added, and conductivity can be improved. The addition amount of the conductive material added to the hydrophobic layer 102 is set to be equal to or less than the upper limit of intake. In addition, the conductive material to be added to the hydrophobic layer 102 is in a state where it can be decomposed in the body or excreted.

Since the edible conductive structure according to the embodiment includes the hydrophobic layer 102, water does not enter the inside of the edible conductive structure even in water, and the conductivity can be maintained, and the thickness can be increased by stacking the conductive layer 101, and the conductivity can be improved.

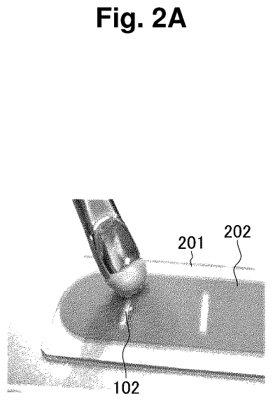

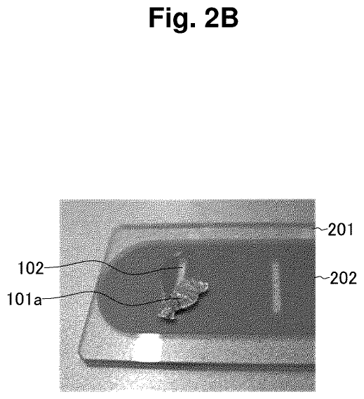

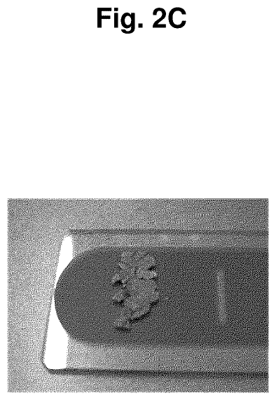

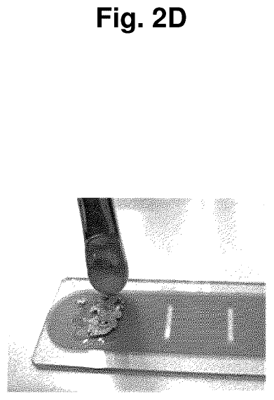

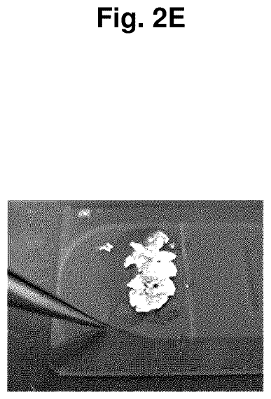

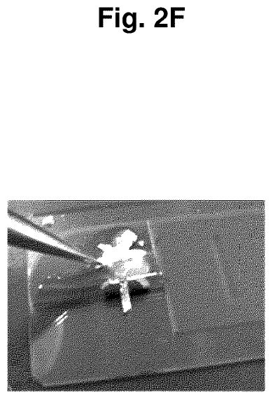

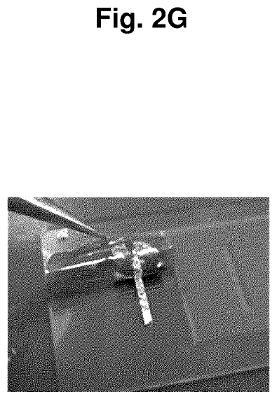

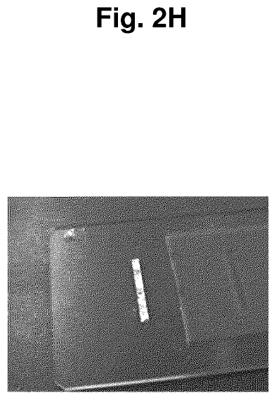

Next, manufacture of the edible conductive structure according to the embodiment will be described with reference to FIGS. 2A to 2H.

First, as illustrated in FIG. 2A, a patterned masking tape 202 is attached to a substrate 201 or the like composed of glass or the like. On the masking tape 202, a rectangular slit pattern of 1 mm×1 cm in plan view is formed by patterning. Next, the substrate 201 is heated (100° C.), and the oleogel is applied along the pattern of the masking tape 202 with a pen to which the oleogel is attached at the tip to form the hydrophobic layer 102.

Next, as illustrated in FIG. 2B, a metal foil 101a such as gold foil or silver foil is attached. The metal foil 101a may have a sheet shape or a flake shape, and is spread so as not to form a gap as illustrated in FIG. 2C. By attaching the metal foil 101a, a conductive layer is formed on the hydrophobic layer 102 formed along the pattern of the masking tape 202.

Next, as illustrated in FIG. 2D, the oleogel is applied again, a metal foil is further attached, and these processes are repeated, and finally, the oleogel is applied so that the surface layer becomes a hydrophobic layer. Thereafter, as illustrated in FIGS. 2E, 2F, and 2G, the masking tape is separated. As a result, as illustrated in FIG. 2H, a wiring-shaped edible conductive structure is formed.

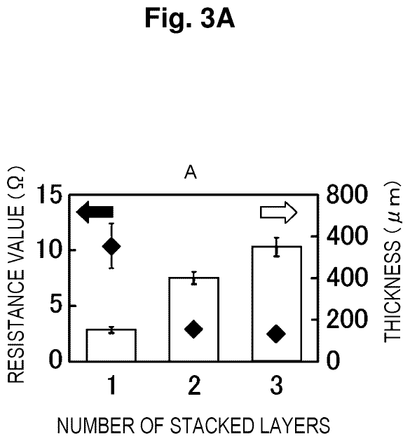

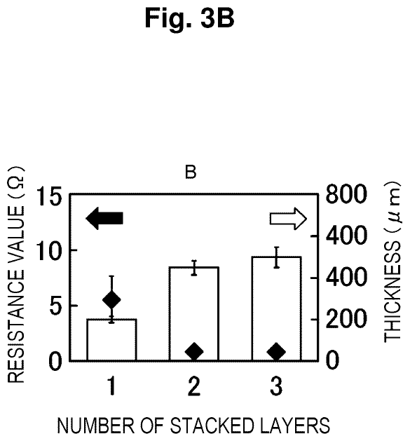

As described above, the relationship between the resistance value and the thickness of the wiring-shaped edible conductive structure (rectangular shape of 1 mm×1 cm in plan shape) produced using the edible gold foil or the edible silver foil and the number of stacked layers is illustrated in FIGS. 3A and 3B. FIG. 3A illustrates the results when an edible gold foil is used, and FIG. 3B illustrates the results when an edible silver foil is used.

As illustrated in FIGS. 3A and 3B, as the number of stacked layers increases, the resistance value decreases, and improvement in conductivity can be confirmed. The conductivity is approximately 103-104 S/m. The conductivity changes depending on the thickness of the metal foil to be used and the shape of the sheet (such as breakage in the sheet). The thicker the metal foil serving as the conductive layer and the less the breakage, the more the conductivity is improved. Even when the size of the metal foil is small with respect to the pattern, the metal foil is partially bonded by the hydrophobic layer made of a sticky hydrophobic material such as oleogel, and the metal foils can be brought into contact with each other, so that conductivity can be maintained.

Incidentally, for example, in the case of a gold thin film having a thickness of about 100 nm formed on a glass substrate by a sputtering method or a vapor deposition method, the contact angle of water is about from hydrophilic to slightly water-repelling. On the other hand, in the case of a gold foil having a thickness of about 300 nm, if there is breakage, water may enter, leading to disconnection. When water drops are dropped on the gold foil, water may pass through the breakage in the foil, and water may spread below the foil.

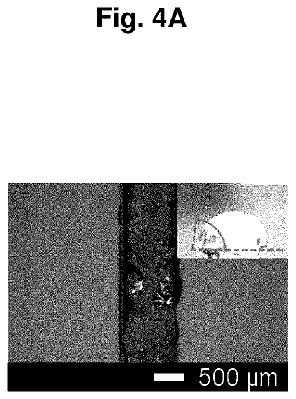

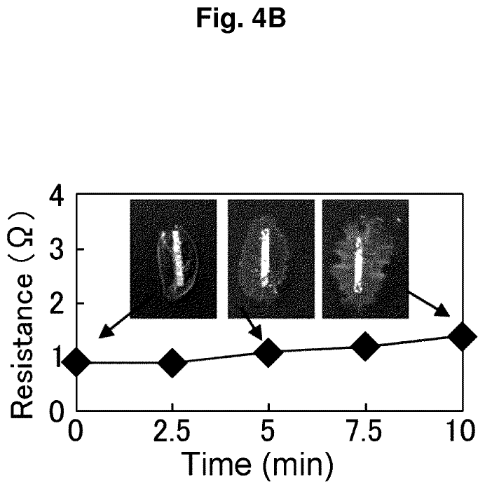

In contrast to the above, in the “structure in which the hydrophobic material and the conductive material are stacked”, even when there is breakage in the metal foil, the metal foil is supported by the hydrophobic material, so that water is less likely to enter. As illustrated in FIG. 4A, in the wiring structure by the edible conductive structure according to the embodiment, the contact angle on the wiring structure is 82°, water does not permeate, and the wiring structure is waterproof. Since this wiring structure is expected not to be disconnected even in water, the wiring structure was produced on the sheet substrate made of gelatin, and a change in resistance value when the wiring structure was immersed in water was measured (FIG. 4B). It can be confirmed that there is no significant change in the resistance value even in water. On the other hand, it can be confirmed that the sheet substrate made of gelatin swells by water absorption.

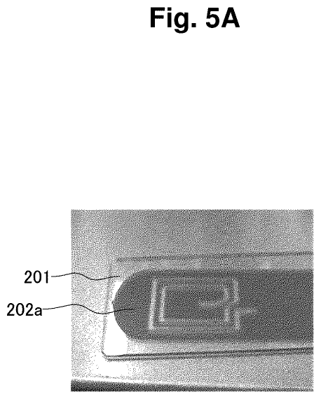

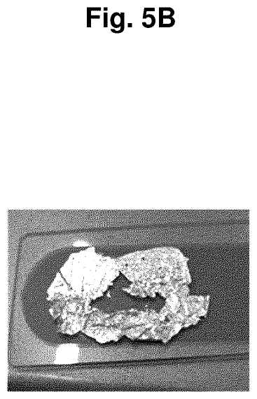

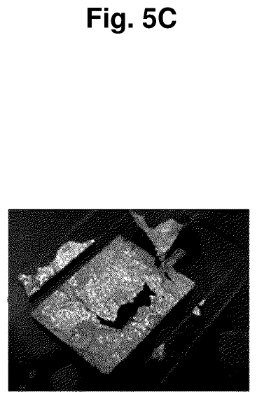

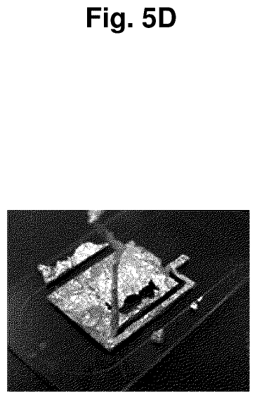

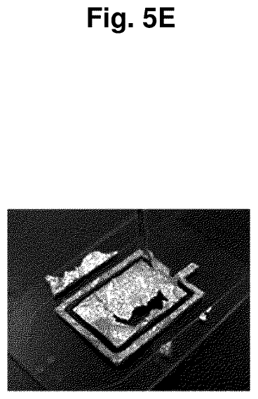

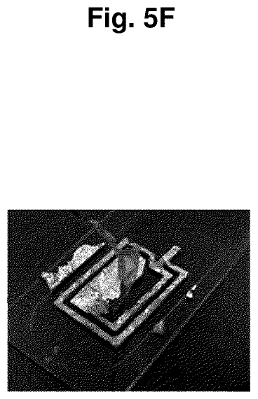

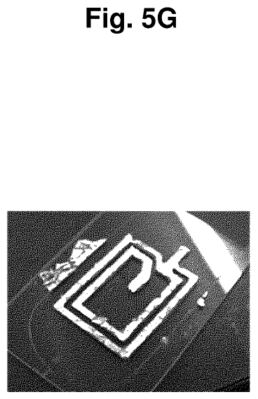

Next, a method for manufacturing an electronic component (coil) to which the edible conductive structure according to the embodiment of the present invention is applied will be described with reference to FIGS. 5A to 5G.

First, as illustrated in FIG. 5A, a patterned masking tape 202a is attached to the substrate 201 or the like composed of glass or the like. On the masking tape 202a, a rectangular slit of 1 mm×1 cm in plan view is formed in a coil shape having a size of 1.4 cm×1.8 cm. Next, the substrate 201 is heated (100° C.), and the oleogel is applied along the pattern of the masking tape 202a with a pen to which the oleogel is attached at the tip to form the hydrophobic layer.

Next, as illustrated in FIG. 5B, a metal foil such as gold foil or silver foil is attached. The metal foil may have a sheet shape or a flake shape, and is spread so as not to form a gap as illustrated in FIG. 5C. By attaching the metal foil, a conductive layer is formed on the hydrophobic layer formed along the pattern of the masking tape 202a.

Next, the oleogel is applied again, a metal foil is further attached, and these processes are repeated, and finally, the oleogel is applied so that the surface layer becomes a hydrophobic layer. Thereafter, as illustrated in FIGS. 5C, 5D, and 5E, the masking tape is separated. As a result, as illustrated in FIG. 5F, an edible conductive structure having a planar coil shape is formed. The inductance of the produced edible conductive structure having a planar coil shape was 0.1 μH.

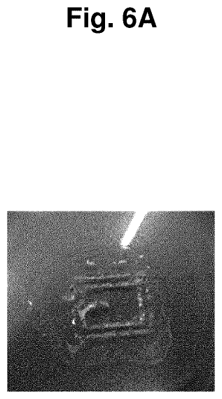

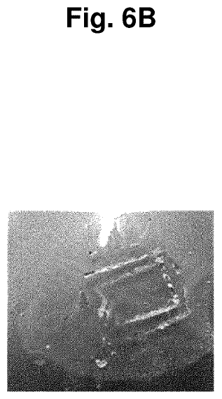

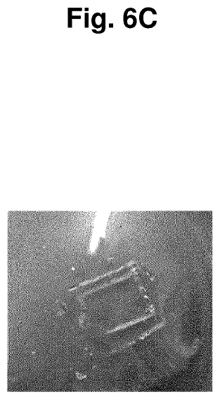

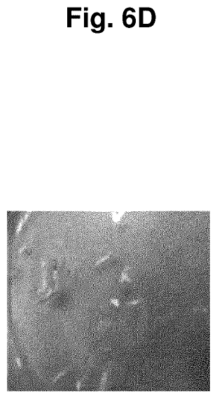

In addition, the edible conductive structure according to the embodiment can be gradually decomposed in water by being formed on an edible substrate such as gelatin. FIGS. 6A to 6D illustrate the results of forming a coil having the edible conductive structure described with reference to FIGS. 5A to 5G on a sheet substrate made of gelatin (gelatin substrate), immersing and leaving the resultant in water. FIG. 6B illustrates a state after leaving for 5 minutes and a state after leaving for 10 minutes. In addition, FIG. 6D illustrates a state of stirring after leaving for 10 minutes. As illustrated in FIGS. 6A to 6D, by leaving in water for about 10 minutes, the pattern of the coil wiring collapses as the gelatin substrate swells. In addition, by stirring, the gelatin substrate further collapses, and the wiring is also shredded.

As described above, by forming the edible conductive structure according to the embodiment on the edible substrate such as gelatin, the edible conductive structure can be shredded and decomposed by an external force such as swelling or stirring of the substrate. In a case where the size of the electronic component is large, there is a possibility of causing blockage in the gastrointestinal tract even when an edible material is used, but such a risk can be further reduced when the component can be shredded and decomposed.

As described above, according to embodiments of the present invention, since the conductive layer including an edible conductive material and the hydrophobic layer including an edible hydrophobic material are alternately stacked, it is possible to provide an edible conductive structure having both waterproofness and conductivity, which can be used in the body or in the environment.

With the edible conductive structure according to embodiments of the present invention, for example, a capacitor or an antenna coil is produced, an RLC circuit is formed, and a change in resonance frequency is read from the outside of the body by magnetic coupling, so that it can be used to estimate the state in the body. In addition, the edible conductive structure according to embodiments of the present invention can be applied to a split-ring resonator, an inverted-F antenna, a dipole antenna, an RFID tag (chipless RFID tag) that does not use an IC chip, and the like, and can be used to evaluate characteristics of a reflected wave and a transmitted wave of a radio wave emitted from the outside of the body and estimate a state in the body.

In addition, since the edible conductive structure according to the present invention is not limited to the inside of the body and uses a food material, the use for measurement in water or soil can be considered by considering metal species while considering the use environment and biological effects. For example, measurement of molecules, ions, pH, and the like in the environment such as water quality investigation, hydroponic cultivation, aquaculture, and soil contamination can be considered. In addition, the edible conductive structure according to embodiments of the present invention can be applied to monitoring of a production line of food and livestock, freshness management of food and drink, logistics management, sales management, and disposal management.

Note that the present invention is not limited to the embodiment described above, and it is obvious that many modifications and combinations can be made by those skilled in the art within the technical idea of the present invention.

### REFERENCE SIGNS LIST

101 Conductive layer
102 Hydrophobic layer

1-6. (canceled)

7. An edible conductive structure comprising:
one or more conductive layers that each include an edible conductive material; and
one or more hydrophobic layers that each include an edible hydrophobic material, wherein the one or more conductive layers and the one or more hydrophobic layers are alternately stacked.

8. The edible conductive structure according to claim 7, wherein the edible hydrophobic material is composed of an organogel.

9. The edible conductive structure according to claim 7, wherein each of the one or more hydrophobic layers is composed of the edible hydrophobic material and a conductive material.

10. The edible conductive structure according to claim 9, wherein the conductive material is a powder of at least one of activated carbon, magnesium, zinc, iron, copper, silver, or gold.

11. The edible conductive structure according to claim 9, wherein the edible hydrophobic material is a fat.

12. The edible conductive structure according to claim 11, wherein the fat is one of fat of meat, lard, corn oil, soybean oil, rapeseed oil, chocolate, butter, or margarine.

13. The edible conductive structure according to claim 7, further comprising a substrate on which the one or more conductive layers and the one or more hydrophobic layers are disposed, wherein the substrate is composed of an edible material.

14. The edible conductive structure according to claim 13, wherein the substrate is capable of decomposition in water.

15. An edible conductive structure comprising:
an edible substrate; and
a multilayer stack made of alternating stacked edible conductive layers and edible hydrophobic layers, wherein the multilayer stack is disposed on the edible substrate.

16. The edible conductive structure according to claim 15, wherein the edible hydrophobic layers are each made of an organogel.

17. The edible conductive structure according to claim 15, wherein each of the edible hydrophobic layers is composed of an edible hydrophobic material and a conductive material.

18. The edible conductive structure according to claim 17, wherein the conductive material is a powder of at least one of activated carbon, magnesium, zinc, iron, copper, silver, and gold.

19. The edible conductive structure according to claim 17, wherein the edible hydrophobic material is a fat.

20. The edible conductive structure according to claim 19, wherein the fat is one of fat of meat, lard, corn oil, soybean oil, rapeseed oil, chocolate, butter, or margarine.

21. The edible conductive structure according to claim 15, wherein the edible substrate is decomposable in water.

22. The edible conductive structure according to claim 15, wherein a uppermost layer of the multilayer stack and a lowermost layer of the multilayer stack are each one of the edible hydrophobic layers.
